(19) United States
(12) Patent Application Publication
(10) Pub. No.: US 2026/0306009 A1
(43) Pub. Date: Oct. 8, 2026

(54) ELECTROCOCHLEOGRAPHY-BASED CLASSIFICATION

(71) Applicant: THE UNIVERSITY OF MELBOURNE, Victoria (AU)

(72) Inventors: Zachary Mark Smith, Pymble, NSW (AU); Aaron Collins, Carlton, VIC (AU); Christofer William Bester, Perth, WA (AU); Ryan Orin Melman, South Coogee, NSW (AU); Stephen O'Leary, Mont Albert, VIC (AU)

(21) Appl. No.: 19/481,667

(22) PCT Filed: May 20, 2024
(86) PCT No.: PCT/IB2024/054897

§ 371 Date: Nov. 5, 2025

Related U.S. Application Data
(60) Provisional application No. 63/504,490, filed on May 26, 2023.

(51) Int. Cl. A61B 5/388; A61B 5/00; A61B 5/25; A61N 1/05; A61N 1/36; A61N 1/372

(52) U.S. Cl. CPC A61B 5/388; A61B 5/25; A61B 5/6847; A61B 5/7264; A61N 1/0541; A61N 1/36039; A61N 1/372

(57) ABSTRACT

ECochG signals are received and used to classify a state of an inner ear into an appropriate category of a plurality of categories. Each category of the plurality of categories corresponds to a respective feedback of a plurality of feedback, and a feedback of the plurality of feedback is selected and output based on the feedback corresponding to the appropriate category in which the state of the inner ear is classified, thereby indicating the specific state of the inner ear.

### BACKGROUND

### Field of the Invention

The present invention relates generally to monitoring electrocochleography (ECochG) signals of a medical device recipient.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatuses, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, one or more non-transitory computer readable media are provided. The one or more non-transitory computer readable media include instructions that, when executed by processing circuitry, cause the processing circuitry to receive an electrocochleography (ECochG) signal associated with a recipient, classify a state of an inner ear of the recipient into an appropriate category of a plurality of categories based on at least one characteristic of the ECochG signal, and output feedback in response to classifying the state of the inner ear into the appropriate category.

In another aspect, a method is provided. The method includes delivering acoustic stimuli to an ear of a recipient, recording a plurality of electrocochleography (ECochG) signals evoked at the ear of the recipient in response to delivery of the acoustic stimuli, and classifying a state of an inner ear of the recipient into a category of a plurality of categories based on a characteristic of the plurality of ECochG signals.

In yet another aspect, a system is provided. The system includes an output device, a network interface for communication with a medical device having a plurality of electrode contacts, a memory storing instructions thereon, and processing circuitry configured to execute the instructions stored on the memory to receive a plurality of electrocochleography (ECochG) signals during implantation of the plurality of electrode contacts into a recipient and classify a state of an inner ear of the recipient into an appropriate category of a plurality of categories based on a characteristic of the plurality of ECochG signals. Each category of the plurality of categories is associated with feedback of a plurality of feedback. The processing circuitry is also configured to execute the instructions stored on the memory to instruct the output device to output appropriate feedback of the plurality of feedback based on the appropriate feedback corresponding to the appropriate category.

### BRIEF DESCRIPTION OF THE DRAWINGS

Embodiments of the present invention are described herein in conjunction with the accompanying drawings, in which:

FIG. 1A is a graph illustrating a cochlear microphonic (CM) magnitude associated with an Electrocochleography (ECochG) signal captured during insertion of a stimulating assembly into an inner ear of a recipient;

FIG. 1B is a graph illustrating another CM magnitude associated with an ECochG signal captured during insertion of a stimulating assembly into another inner ear of a recipient;

FIG. 2 is a schematic diagram illustrating a cochlear implant system implanted in a head of a recipient and an ECochG monitoring system associated with the cochlear implant system, in accordance with certain embodiments presented herein;

FIG. 3 is a schematic diagram illustrating the cochlear implant system and the ECochG monitoring system of FIG. 2, showing a side view of the head of the recipient, in accordance with certain embodiments presented herein;

FIG. 4 is a schematic diagram illustrating the cochlear implant system and the ECochG monitoring system of FIG. 2, showing components of the cochlear implant system without the recipient's head for purposes of clarity, in accordance with certain embodiments presented herein;

FIG. 5 is a block diagram of the cochlear implant system and the ECochG monitoring system of FIG. 2, in accordance with certain embodiments presented herein;

FIG. 6 is a flowchart of a method of performing an ECochG signal processing operation, in accordance with certain embodiments presented herein;

FIG. 7 is a flowchart of a method of resetting a measurement value of ECochG signals, in accordance with certain embodiments presented herein;

FIG. 8 is a flowchart of a method of providing feedback based on CM magnitudes, in accordance with certain embodiments presented herein;

FIG. 9 is a flowchart of another method of providing feedback based on CM magnitudes, in accordance with certain embodiments presented herein;

FIG. 10 is a flowchart of yet another method of providing feedback based on CM magnitudes, in accordance with certain embodiments presented herein;

FIG. 11 is a flowchart of a method of classifying a state of an inner ear using ECochG signals, in accordance with certain embodiments presented herein; and

FIG. 12 is a schematic diagram illustrating a vestibular stimulator system with which aspects of the techniques presented herein can be implemented.

### DETAILED DESCRIPTION

Auditory/hearing device recipients/users suffer from different types of hearing loss (e.g., conductive and/or sensorineural) and/or different degrees/severity of hearing loss. However, it is now common for many recipients to retain some residual natural hearing ability (residual hearing) after receiving the hearing device. That is, hearing device recipients often retain at least some of their natural ability to hear sounds without the aid of their hearing prosthesis. For example, cochlear implants can now be implanted in a manner that preserves at least some of the recipient's cochlear hair cells and the natural cochlear function, particularly in the lower frequency regions of the cochlea.

Electrocochleography (ECoG or ECochG) refers to a clinical measurement technique that involves the delivery of acoustic stimuli to a recipient's cochlea and recording one or more responses (ECochG responses or ECochG signals) of the cochlea to the acoustic stimulus. For example, during certain ECochG testing procedures, preselected/predetermined clicks or tones are delivered acoustically to the inner ear of recipient, and an ECochG response/signal is recorded by using an electrode in or near the recipient's middle ear or inner ear. An example operation in which ECochG can be performed is during insertion of a stimulating assembly into the cochlea, during which an ECochG signal can be recorded from one or more electrodes (e.g., the most apical electrode) of the stimulating assembly. The recorded ECochG signals can then be used to, for example, assess the hearing of the recipient and operate a hearing device accordingly, such as to provide stimulation via output signals (e.g., for non-functioning regions of the inner ear, for sounds above a threshold frequency), while preserving remaining functionality of the cochlea of the recipient.

As used herein, an ECochG signal can include one or a plurality of different characteristics, such as stimulus related electrical potentials (e.g., a set of ECochG responses) that include a cochlear microphonic (CM), a cochlear summating potential (SP), and an auditory nerve neurophonic (ANN)/auditory nerve Action Potential (AP), where these parameters are measured/recorded independently or in various combinations in response to delivery of an acoustic stimulus to the inner ear. In general, the ECochG signal recording may be completed within a short time period (e.g., a few milliseconds (ms) after the initial delivery of the acoustic stimuli) and does not have to wait until after completion of the acoustic stimuli.

The CM is an alternating current (AC) voltage that mirrors the waveform of the acoustic stimulus at low to moderate levels of acoustic stimulation. The CM is generated by the outer hair cells of the organ of Corti and is dependent on the proximity of the recording electrode to the stimulated hair cells. In general, the CM is proportional to the displacement of a basilar membrane. Different properties of the CM, including the CM magnitude/amplitude and/or the CM latency or delay (e.g., time between acoustic stimulation output and observed CM response measurement), can be monitored.

The SP is the direct current (DC) component of the EcochG response containing contributions from outer hair cells, inner hair cells, and/or spiral ganglion neurons as they, or their cellular targets (e.g., in case of neurons), are moved in conjunction with the basilar membrane (i.e., reflects the time-displacement pattern of the cochlear partition in response to the stimulus envelope). The SP is the stimulus-related potential of the cochlea and can be seen as a DC (unidirectional) shift in the cochlear microphonic baseline. The direction of this shift (i.e., positive or negative) is dependent on a complex interaction between stimulus parameters and the location of the recording electrode.

The ANN represents phase-locked firing of auditory nerve fibers in response to the acoustic stimuli, and the ANN appears as an alternating current voltage with maximal energy as twice the stimulus frequency for acoustic stimuli that include pure tones. The ANN also includes a magnitude and a latency. The magnitude of the ANN reflects the number of nerves that are firing, while the latency of the ANN is measured as the time to the first negative deflection.

In conventional arrangements, it is difficult to utilize the ECochG signals to perform a corresponding action in response. For instance, it may be difficult to observe the ECochG signals (e.g., identify a change in the ECochG signals), assess the recipient based on the ECochG signals, determine a corresponding action to be performed based on the assessment of the recipient (e.g., the mitigate the change in the ECochG signals), and perform the corresponding action. Indeed, manual observation of the ECochG signals may be limited, especially when performed simultaneously with a surgical operation, such as insertion of a stimulating assembly into the cochlea. As an example, more nuanced or subtle features of the ECochG signals may not be detectable by a user (e.g., a technician, a surgeon). As another example, it is difficult to determine a cause of an observed ECochG signal, such as whether a change in a feature of the ECochG signals is caused by inherent function of the recipient's cochlear anatomy (e.g., due to outer hair cell (OHC) health) or by a surgical operation being performed. Thus, a subsequent action may not be identified or effectively performed based on the ECochG signal monitoring.

Accordingly, embodiments of the present disclosure are directed to an ECochG monitoring system configured to receive ECochG signals, classify a state of the inner ear into an appropriate category selected from multiple different available categories based on the ECochG signals, and provide feedback to indicate the category in which the state of the inner ear is classified. By way of example, the ECochG monitoring system extracts various characteristics, such as the CM, the SP, and/or the ANN, and classifies the state of the inner ear into an appropriate category based on the characteristics, such as based on different permutations or combinations of trends of the characteristics. Because there are multiple different available categories of the state of the inner ear, the ECochG monitoring system selects specific feedback to be output based on the corresponding state of the inner ear. Thus, the user is readily able to determine the particular state of the inner ear (e.g., by distinguishing the determined state of the inner ear from other possible or available states of the inner ear) based on the output feedback. The user can then perform a suitable action in response, such as to retract or otherwise manipulate the position of the stimulating assembly in the cochlea. Indeed, the feedback provided by the ECochG monitoring system can indicate a potential cause of certain received ECochG signals to better inform the user and prompt the user to respond more appropriately. As such, an assessment of a state of the recipient's inner ear and/or a performance of a corresponding action based on the assessment is improved.

There are a number of different types of devices in/with which embodiments of the present invention may be implemented. Merely for ease of description, the techniques presented herein are primarily described with reference to a specific device in the form of a cochlear implant system. However, it is to be appreciated that the techniques presented herein may also be partially or fully implemented by any of a number of different types of devices, including consumer electronic device (e.g., mobile phones), wearable devices (e.g., smartwatches), hearing devices, implantable medical devices, wearable devices, etc. consumer electronic devices, wearable devices (e.g., smart watches, etc.), etc., that have the ability to record ECochG signals. As used herein, the term “hearing device” is to be broadly construed as any device that acts on an acoustical perception of an individual, including to improve perception of sound signals, to reduce perception of sound signals, etc. In particular, a hearing device can deliver sound signals to a user in any form, including in the form of acoustical stimulation, mechanical stimulation, electrical stimulation, etc., and/or can operate to suppress all or some sound signals. As such, a hearing device can be a device for use by a hearing-impaired person (e.g., hearing aids, middle ear auditory prostheses, bone conduction devices, direct acoustic stimulators, electro-acoustic hearing prostheses, auditory brainstem stimulators, bimodal hearing prostheses, bilateral hearing prostheses, dedicated tinnitus therapy devices, tinnitus therapy device systems, combinations or variations thereof, etc.), a device for use by a person with normal hearing (e.g., consumer devices that provide audio streaming, consumer headphones, earphones, and other listening devices), a hearing protection device, etc. In other examples, the techniques presented herein can be implemented by, or used in conjunction with, various implantable medical devices, such as vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

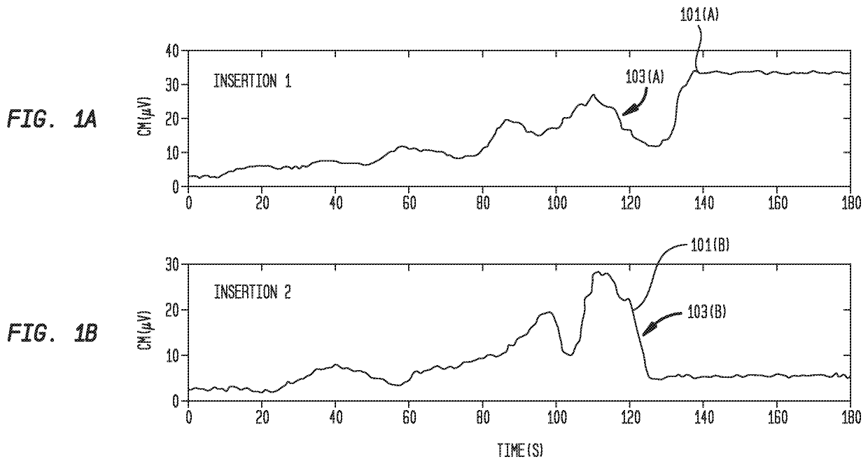

With the preceding in mind, FIG. 1A is a graph illustrating part of an ECochG signal, namely a CM magnitude 101(A), as a function of time for the surgical insertion of a first stimulating assembly into a first cochlea. FIG. 1B is a graph illustrating a CM magnitude 101(B), as a function of time for the surgical insertion of a second stimulating assembly into a second cochlea. The CM magnitudes 101(A) and 101(B) shown in FIGS. 1A and 1B, respectively, represent recordings made from the most apical electrode for a 500 Hertz (Hz) probe stimulus. Note that in both cases there are several rises and falls in the CM magnitudes over the course of the insertions, and that it is difficult to efficiently identify the rises and falls and/or determine the cause of change in CM magnitude from these recordings alone. By way of example, at a region 103(A) of the graph illustrated in FIG. 1A, the CM magnitude 101(A) is decreasing, and at a region 103(B) of the graph illustrated in FIG. 1B, the CM magnitude 101(B) is also decreasing. However, the cause of the drop of the CM magnitude 101(A) at the region 103(A) is different from the cause of the drop of the CM magnitude 101(B) at the region 103(B). Thus, a suitable action to perform with respect to the CM magnitude 101(A) can be different from a suitable action to perform with respect to the CM magnitude 101(B). In this manner, it can be difficult to assess a state of the inner ear and perform a corresponding action by merely visually observing the change in the CM magnitude, such as by determining that a drop in the CM magnitude exists.

The techniques presented herein operate to determine a state of the inner ear based on ECochG signals. For example, a trend or value of the CM magnitude is determined and compared with a threshold. Indeed, there can be multiple, different thresholds for comparison, and one of many different potential states of the inner ear can be determined based on the CM magnitude relative to the thresholds. Particular feedback is then output to prompt a user activity based on the determined state of the inner ear. In this manner, a specific state of the inner ear can be more accurately determined and indicated to prompt performance of a suitable action.

For instance, the position or relative movement of the stimulating assembly within a cochlea can be tracked, for example, by: impedance monitoring of the electrodes, analysis of the phase/latency of the ECochG signal, visual tracking (surgical microscope), radiographic video imaging, such as fluoroscopy, and/or other techniques. The recorded ECochG signals and the position information, can be used to determine the state of the inner ear at the region of the inner ear in which the stimulating assembly is positioned and corresponding acoustic stimuli is provided. As an example, the recorded ECochG signals and the position information can be used to determine varying degrees of functionality at the different regions of the inner ear. As another example, the recorded ECochG signals and the position information can indicate whether the positioning of the stimulating assembly is desirable, or whether the positioning should be adjusted. By improving the interpretation of recorded ECochG signals and corresponding classification of the state of the inner ear, implantation of the stimulating assembly, preservation of the residual hearing of the recipient, or other aspects can be improved.

As used herein, the “position” of the stimulating assembly (e.g., an electrode) generally refers to the insertion depth (e.g., angular insertion depth) of the stimulating assembly in the inner ear (e.g., cochlea). However, the “position” of the stimulating assembly can also include the relative proximity of the stimulating assembly to a wall of the inner ear (e.g., modiolar proximity, lateral wall proximity, etc.), distance from the mid-modiolar axis, or other information relating to the placement or position of one or more parts of the stimulating assembly.

In general, the techniques presented herein record ECochG signals and stimulating assembly position information and then analyze this information to determine a state of the inner ear at the stimulating assembly position. For example, the state of the inner ear may indicate local anatomy variations as the stimulating assembly moves through the inner ear, a function of the cochlea, an unexpected change in acoustic responsiveness of the cochlea, and so forth. In accordance with certain embodiments presented herein, a system iteratively records (e.g., continuously, periodically, etc.) ECochG signals from a “recording site” within the inner ear (e.g., cochlea). In these embodiments, the stimulating assembly includes an elongate carrier member having a plurality of longitudinally spaced electrodes. One or more of the electrodes of the stimulating assembly are used to record ECochG signals.

In certain embodiments, the recording site is fixed to the most apical electrode of the moving stimulating assembly, meaning that the position/location (e.g., insertion depth, modiolar proximity, etc.) of the recording site will change over time as the stimulating assembly is progressively inserted into the inner ear or otherwise adjusted, but that the electrode used to make the recording does not change (i.e., the recording site remains the most apical electrode of the stimulating assembly as the most apical electrode is moved via movement of the stimulating assembly). In alternative embodiments, the recording site is a fixed position (e.g., a predetermined insertion depth within the inner ear), meaning that the position/location of the recording site will remain substantially constant/fixed over time as the stimulating assembly is progressively inserted into the inner ear or otherwise adjusted, but that the electrode used to make the recording will change over time (e.g., the electrode changes to be the electrode most proximate the predetermined constant insertion depth within the inner ear). In such embodiments, the position of the recording site can be, for example, near the base of the cochlea or another fixed location with a robust ECochG response (e.g., will not record at the cochlea base if the ECochG signal is very small or absent).

In accordance with these embodiments, for each ECochG signal recording, the inner ear position of the electrode corresponding to a recording, and potentially the time at which the recording was made, is also obtained/recorded and associated with the corresponding ECochG signal recording. This information is then used by the system to analyze the ECochG signals in a relative manner to classify the state of the inner ear into an appropriate category. For example, the ECochG signal is monitored over time to determine a trend, and the trend is further analyzed to classify the state of the inner ear. As such, the techniques presented herein can facilitate interpretation of ECochG signals, the resulting implications thereof, and actions to be performed, if needed. For instance, users can be notified based on the category in which the state of the inner ear is classified to improve surgical interventions to maximize/balance hearing preservation and stimulating assembly insertion depth. The techniques presented herein therefore facilitate the creation of algorithms that automate the interpretation of ECochG signals and provide users with more meaningful information to guide their decision process and, for example, maximize preservation of residual hearing and overall outcomes.

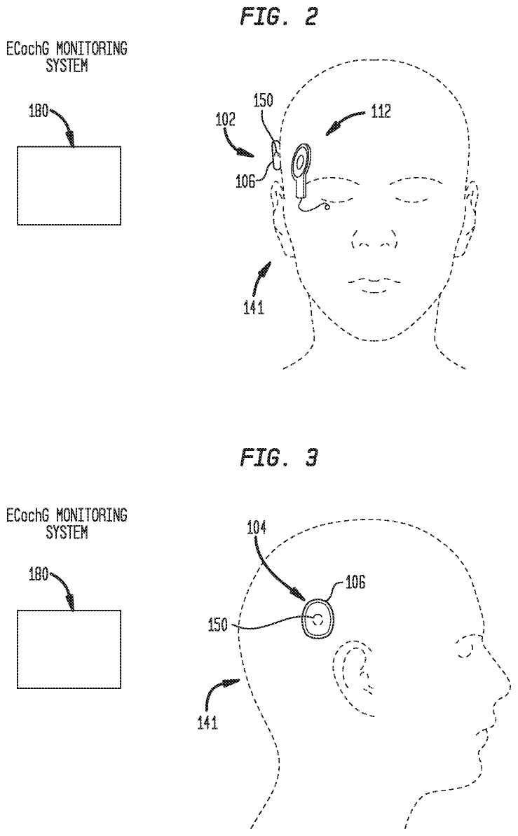

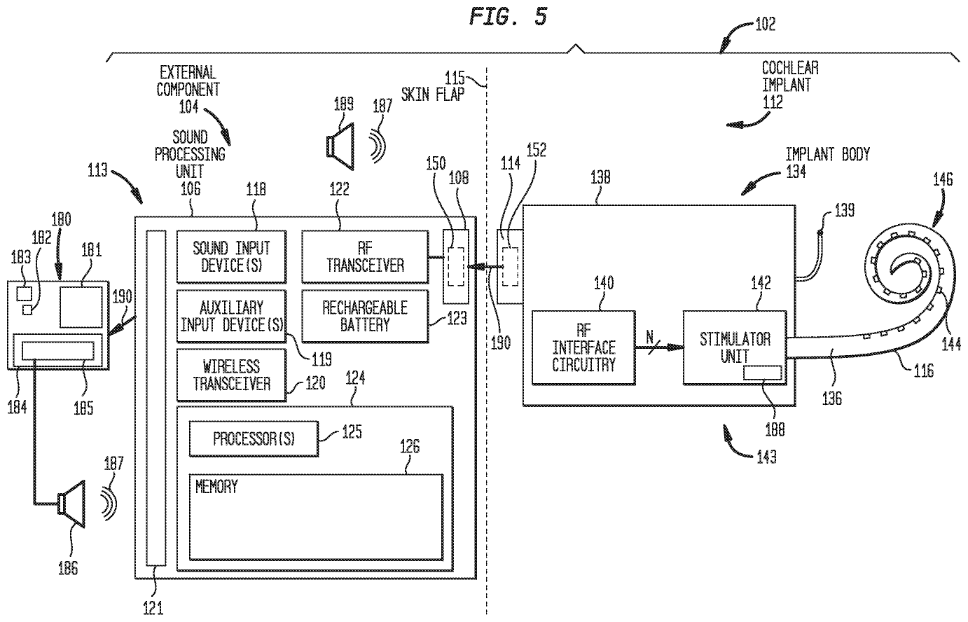

Referring initially to FIGS. 2-5, which are generally described together for ease of description, an example cochlear implant system 102 can be implanted in a head 141 of a person, animal, or other recipient (collectively and generally referred to herein as a “recipient”). The cochlear implant system 102 includes an external component 104 and an implantable component 112. The implantable component 112 is sometimes referred to as a “cochlear implant.” The cochlear implant system 102 operates with an ECochG monitoring system 180. The ECochG monitoring system 180, which is shown in greater detail in FIG. 5, could be implemented by an suitable computing system, environment, or configuration including, but are not limited to, personal computers, server computers, hand-held devices, laptop devices, desktop devices, multiprocessor systems, microprocessor-based systems, programmable consumer electronics (e.g., smart phones), remote control units, network PCs, minicomputers, mainframe computers, tablets, distributed computing environments that include any of the above systems or devices, and the like. The ECochG monitoring system 180 and the cochlear implant system 102 (e.g., a sound processing unit 106 or the implantable component 112) wirelessly communicate via a communication link, such as a short-range communication (e.g., a Bluetooth link, Bluetooth Low Energy (BLE) link, a proprietary link, etc.). The ECochG monitoring system 180 can be include any suitable arrangement of components inserted within the ear of the recipient or disposed external to the recipient.

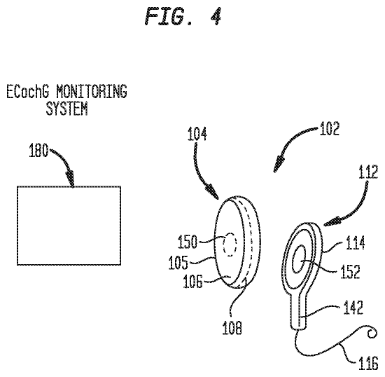

FIG. 2 is a schematic diagram illustrating the implantable component 112 implanted in the head 141 of the recipient, while FIG. 3 is a schematic diagram illustrating the external component 104 configured to be positioned adjacent the head 141 of the recipient. FIG. 4 includes another schematic view of the cochlear implant system 102, including both the external component 104 and the implantable component 112, but without the recipient's head being shown for purposes of clarity. FIG. 5 is a block diagram illustrating further details of the cochlear implant system 102 and the ECochG monitoring system 180 associated with the cochlear implant system 102, in accordance with certain embodiments presented herein. For ease of illustration, FIGS. 2, 3, 4, and 5 will generally be described together.

As noted, the cochlear implant system 102 includes an external component 104 that is configured to be directly or indirectly attached to the body of the recipient and an implantable component 112 configured to be implanted in the recipient. In the examples of FIGS. 2-5, the external component 104 includes a sound processing unit 106, which is an off-the-ear (OTE) sound processing unit sometimes referred to as an “OTE component.” The sound processing unit 106 is configured to send data and power to the implantable component 112 as described herein.

In the arrangement shown in FIGS. 2-5, the sound processing unit 106 includes a generally cylindrically shaped housing 105, which is configured to be magnetically coupled to the recipient's head 141. For example, the sound processing unit 106 can include an integrated external magnet 150 configured to be magnetically coupled to an implantable magnet 152 in the implantable component 112. The sound processing unit 106 also includes an integrated external coil 108 that is configured to be wirelessly (e.g., inductively) coupled to an implantable coil 114 of the implantable component 112 as described below. In FIGS. 2-4, the external magnet 150 is shown using dashed lines, indicating it is integrated within the housing 105 of the sound processing unit 106. In FIG. 5, the external magnet 150 and the implantable magnet 152 are shown using dashed lines, indicating the external coil 108 and the implantable coil 114 are disposed around the magnet 150 and magnet 152, respectively.

It is to be appreciated that the arrangement shown in FIGS. 2-5 is merely illustrative and that other arrangements are possible. In particular, the OTE sound processing unit 106 is merely illustrative of the external devices that could operate with the implantable component 112. For example, in alternative examples, the external component may include a behind-the-ear (BTE) sound processing unit or a micro-BTE sound processing unit and a separate external coil assembly. In general, a BTE sound processing unit includes a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

More generally, the OTE sound processing unit 106 is used for communication between the ECochG monitoring system 180 and the implantable component 112. As such, during a surgical procedure, the OTE sound processing unit 106 could be replaced by any other device that is able to communicate with the ECochG monitoring system 180 and the implantable component 112. In certain embodiment, the OTE sound processing unit 106 could be a so-called “surgical processor” having less capabilities than the OTE sound processing unit 106 (e.g., no sound processing logic, etc.). In various embodiments, the communication between the ECochG monitoring system 180 and the OTE sound processing unit 106, or another device operating in place of the OTE sound processing unit 106, could communicate via a wireless or wired connection. In other embodiments, the implantable component 112 could communicate directly (e.g., via a wireless connection) with the ECochG monitoring system 180.

FIG. 5 illustrates the sound processing unit 106, which includes the external coil 108, a wireless transmitter/receiver (transceiver) 120, a charging coil 121, closely-coupled interface circuitry (transceiver) 122, sometimes referred to as a radio-frequency (RF) circuitry 122, at least one rechargeable battery 123, and a processing module 124 that includes one or more processors 125 and a memory device (memory) 126. For example, the processor(s) 125 execute instructions stored on the memory device 126 to instruct the RF transceiver 122 to communicate with the implantable component 112 (e.g., to receive ECochG signal data from the implantable component 112 via the external coil 108), and/or the processor(s) 125 execute instructions stored on the memory device 126 to instruct the wireless transceiver 120 to communicate with the ECochG monitoring system 180 (e.g., to forward ECochG signal data to the ECochG monitoring system 180). In some embodiments, the sound processing unit 106 includes one or more input devices 113, including one or more sound input devices 118 and/or one or more auxiliary input devices 119, which can receive data (e.g., sound data) used to operate the sound processing unit 106, such as to communicate with the implantable component 112 and/or with the ECochG monitoring system 180. The sound processing unit 106 further includes a charging coil 121 and at least one rechargeable battery 123. The at least one rechargeable battery 123 stores power used to enable operation of the sound processing unit 106 (e.g., of the processor(s) 125), and the charging coil 121 provides the power to be stored in the at least one rechargeable battery 123.

The implantable component 112 includes an implant body (main module) 134, a lead region 136, and an intra-cochlear stimulating assembly 116, all configured to be implanted under a skin/tissue of the recipient. The magnets 150 and 152 magnetically couple the external component 104 to the implantable component 112 through the skin/tissue to establish a wireless link between the coils 108 and 114, such as an RF link, an infrared (IR) link, an electromagnetic link, a capacitive and inductive link, and so forth, that may be used to transfer the power and/or data between the external component 104 and the implantable component 112. The implant body 134 generally comprises a hermetically-sealed housing 138 in which an RF interface transceiver 140 and a stimulator unit 142 are disposed. The implant body 134 also includes the internal/implantable coil 114 that is generally external to the housing 138, but which is connected to the RF interface transceiver 140 via a hermetic feedthrough (not shown in FIG. 5).

The stimulating assembly 116 is configured to be at least partially implanted in the recipient's cochlea. The stimulating assembly 116 includes a plurality of longitudinally spaced intra-cochlear electrical stimulating contacts (electrodes) 144 that collectively form a contact or electrode array 146. The stimulating assembly 116 is configured to be inserted into an opening in the recipient's cochlea (e.g., cochleostomy, the round window, etc.) and has a proximal end connected to stimulator unit 142 via the lead region 136 and hermetic feedthrough. The lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

The stimulator unit 142 generates electrical stimulation signals (e.g., current signals), such as based on data provided by the sound processing unit 106 (e.g., by the RF transceiver 122) for delivery to the user's cochlea via one or more of the electrodes 144. In this way, the cochlear implant system 102 electrically stimulates the user's auditory nerve cells, bypassing absent or defective hair cells that normally transduce acoustic vibrations into neural activity, in a manner that causes the recipient to perceive one or more components of the input audio signals (the received sound signals). Additionally, the electrodes 144 are configured to record ECochG signals provided by the cochlea, and the RF interface circuitry 140 is configured to direct the recorded ECochG signals to the sound processing unit 106 and toward the ECochG monitoring system 180.

The ECochG monitoring system 180 includes a user interface 181, one or more processors 182, a network interface (e.g., wireless module) 183, and a memory device (memory) 184 storing ECochG monitoring logic 185. The ECochG monitoring system 180 can also include other components, such as a system bus, component interfaces, a graphics system, a power source (e.g., a battery), among other components. The memory device 184 may include any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors 182 are, for example, microprocessors, microcontrollers, or any other firmware elements, partially or fully implemented with digital logic gates in one or more application-specific integrated circuits (ASICs), partially or fully in software, etc. configured to execute instructions associated with the ECochG monitoring logic 185.

The network interface 183 enables communication with the external component 104 and/or the cochlear implant 112. For example, the network interface 183 can include a wireless module that is similar to wireless transceiver 120, described elsewhere herein, for wireless communication with the external component 104 (or cochlear implant 112, if enabled with a wireless module). Additionally or alternatively, the network interface 183 can provide wired network access. The network interface 183 can support one or more of a variety of communication technologies and protocols, such as ETHERNET, cellular, BLUETOOTH, near-field communication, and RF (Radiofrequency), among others. The network interface 183 can include one or more antennas and associated components configured for wireless communication according to one or more wireless communication technologies and protocols.

The user interface 181 includes, for example, one or more input devices over which the ECochG monitoring system 180 receives input from a user, and one or more output devices by which the ECochG monitoring system 180 is able to provide output to a user. The one or more input devices can include physically-actuatable user-interface elements (e.g., buttons, switches, or dials), touch screens, keyboards, mice, pens, and voice input devices, among others input devices configured to receive a user input. The one or more output devices can include displays, speakers, and printers, among other output devices for presentation of feedback (e.g., visual, audible, or tactile information) to the recipient, a clinician, an audiologist, or other user.

It is to be appreciated that the arrangement for the ECochG monitoring system 180 shown in FIG. 5 is merely illustrative and that aspects of the techniques presented herein can be implemented at a number of different types of systems/devices including any combination of hardware, software, and/or firmware configured to perform the functions described herein. For example, the ECochG monitoring system 180 can be a personal computer (e.g., a desktop or laptop computer), a hand-held device (e.g., a tablet computer), a mobile device (e.g., a smartphone), a surgical system, and/or any other electronic device having the capabilities to perform the associated operations described elsewhere herein.

In accordance with embodiments presented herein, the ECochG monitoring system 180 is configured to record ECochG signals from a recording site, such as while the stimulating assembly 116 is inserted into the recipient's cochlea. More specifically, the ECochG monitoring system 180 is configured to use the electrodes 144 of the electrode array 146 to capture ECochG signals from the cochlea.

In a normal or fully functional ear, an acoustic pressure or sound wave (i.e., a sound signal) is collected by the outer ear and channeled into and through the ear canal. Disposed across the distal end of ear cannel is a tympanic membrane that vibrates in response to sound wave. This vibration is coupled to the oval window through three bones of middle ear. The middle ear bones serve to filter and amplify sound wave, causing the oval window to articulate, or vibrate, in response to vibration of tympanic membrane. This vibration sets up waves of fluid motion of a perilymph within the cochlea to active the cochlea hair cells. Activation of the hair cells causes appropriate nerve impulses to be generated and transferred through the recipient's spiral ganglion cells and auditory nerve to the brain where they are perceived as sound.

As noted above, it is common for hearing device recipients to retain at least part of this normal hearing functionality (i.e., retain at least some residual hearing). Therefore, the cochlea of hearing prosthesis recipient can be acoustically stimulated upon delivery of a sound signal to the recipient's outer ear without the aid of the hearing prosthesis itself. In certain recipients, the normal hearing functionality may be enhanced through the use of an acoustic transducer in or near the outer ear and/or ear canal. In such recipients, the acoustic transducer is used to, for example, filter, enhance, and/or amplify a sound signal which is delivered to the cochlea via the middle ear bones and oval window, thereby creating waves of fluid motion of the perilymph within the cochlea. In other recipients, the normal hearing functionality may be enhanced through the use of a mechanical transducer that is coupled to the individual's bone (e.g., skull, jaw, etc.). In such recipients, the mechanical transducer delivers vibration to the individual's bone, and the vibration is relayed to the cochlea so as to create waves of fluid motion of the perilymph within the cochlea.

As such, an ECochG recording used in accordance with embodiments presented herein can be initiated by the ECochG monitoring system 180. The ECochG recording involves the delivery of acoustic stimuli to the recipient's cochlea and recording one or more responses of the cochlea to the acoustic stimulus. As used herein, acoustic stimuli refer to any type of stimulation that is delivered in a manner so as to set up waves of fluid motion of the perilymph within the cochlea that, in turn, activates the hair cells inside of cochlea. As such, acoustic stimuli for performance of an ECochG recording in accordance with embodiments presented herein may be delivered via a recipient's normal hearing functionality, via an acoustic transducer, via a mechanical transducer, a combination thereof, etc.

FIG. 5 illustrates an embodiment in which an acoustic transducer in the form of an external speaker 186 is configured to deliver an acoustic stimulus 187 to the cochlea of the recipient. FIG. 5 also illustrates that the cochlear implant 112 includes a recording module 188 that is configured to record ECochG signals induced in the cochlea by the acoustic stimulus 187. The recording module 188 may include, for example, sense amplifiers configured to digitally record ECochG signals/responses presented on an input line connected to one or more of the electrodes 144. Data recorded by the sense amplifiers can, in certain embodiments, be stored in a buffer. Additionally or alternatively, a receiver (speaker) 189 is configured to deliver the acoustic stimulus 187 to the ear of the recipient. For example, the receiver 189 is an in-the-ear (ITE) device that is positioned within an ear canal of the recipient (e.g., as a separate component from the external component 104 and from the cochlear implant 112). The receiver 189 could be a stand-alone component that is in wireless connection, for example, the sound processing unit 106, connected to the sound processing unit 106 via wire connection, etc. On example, the receiver 189 is a component of a hearing aid. In such embodiments, the recording module 188 is also configured to record the ECochG signals induced in the cochlea by the acoustic stimulus 187 delivered via the receiver 189.

The RF interface transceivers 122, 140 cooperate to provide ECochG signal data (e.g., the captured ECochG signals, data associated with the captured ECochG signals, such as recording position and/or time information) to the sound processing unit 106, where the ECochG signal data is then provided to the ECochG monitoring system 180. The ECochG signal data is generally represented in FIG. 5 by arrows 190. The processors 182 then analyze the ECochG signal data to classify the state of the inner ear in an appropriate category. The processors 182 can also operate the user interface 181 based on classification of the state of the inner ear to provide feedback that prompts a user (e.g., a surgeon) to perform a corresponding action.

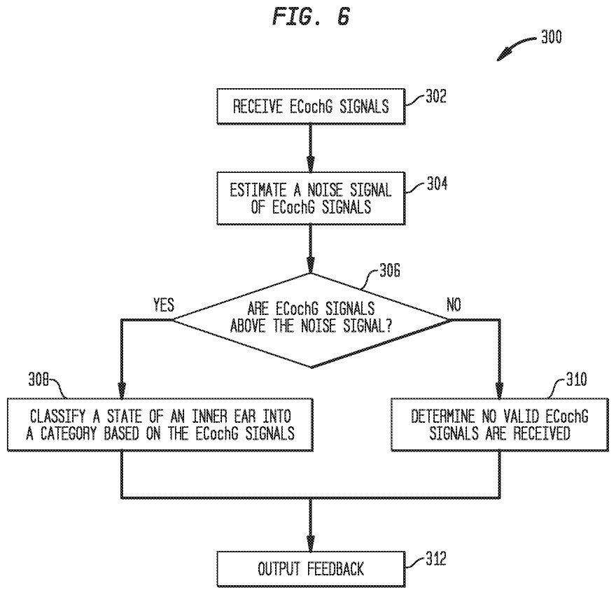

Each of FIGS. 6-11 illustrates a respective method related to ECochG signal processing. In certain embodiments, each method can be performed by a single component or device. For example, the ECochG monitoring system 180 can perform each of the methods. In additional or alternative embodiments, different components or devices can perform different methods and/or different operations within a method. It should be noted that any of the methods can be performed differently than depicted. For instance, a certain operation may not be performed, an additional operation may be performed, and/or any depicted operation may be performed in a different order. Moreover, different methods can be performed in any suitable manner relative to one another, such as at the same time as, independently of, or in response to one another.

FIG. 6 is a flowchart of a method 300 of performing an ECochG signal processing operation, in accordance with certain embodiments presented herein. At 302, recorded ECochG signals are received for processing. For example, acoustic stimuli (e.g., an acoustic tone that has a frequency closer to that at an insertion point than a maximum perceivable sound frequency, or characteristic frequency, of the most apical position of a fully inserted electrode array of a stimulating assembly, such as between 250 Hz and 500 Hz) are delivered to a recipient's cochlea and one or more ECochG signals are recorded, via one or more electrodes positioned in the ear of the recipient, in response to the acoustic stimuli. In some embodiments, different acoustic stimuli (e.g., acoustic tones having different frequencies) are delivered contemporaneously (e.g., in parallel or simultaneously, sequentially, etc.). The acoustic stimuli can be continually or periodically presented (e.g., in short bursts) and in alternating polarity such that a difference between cochlear responses to rarefaction (i.e., low acoustic pressure) and condensation (i.e., high acoustic pressure) can be determined. Additionally or alternatively, acoustic stimuli at a single polarity can be delivered.

At 304, a noise signal of the ECochG signals is estimated. A noise signal generally refers to any signal recording that does not represent the cochlea response signal provided as a result of delivered acoustic stimuli. For example, a noise signal can be caused by electrical interference by adjacent electrical components and/or by a surrounding environment (e.g., body tissue, activation of other neurons of the recipient). Thus, the noise signal should be determined as a part of the processing of ECochG signals to determine cochlea responses more accurately. Noise estimation can be used upon receipt of a significant ECochG signal and/or of a substantial change in ECochG signals.

In some embodiments, direct time domain measurement dispersion estimation can be performed to estimate a noise signal. As an example, the noise signal is estimated based on a dispersion (e.g., standard deviation, median absolute difference) of baseline signals received when no stimulus is provided, a dispersion of mean differences between baseline signals received at different times when no stimulus is provided, and/or a minimum of either a dispersion at a beginning or a dispersion at end of an ECochG signal (e.g., first 1.5 ms of an ECochG signal, last 1.5 ms of an ECochG signal) when no stimulus is believed to be provided. In additional or alternative embodiments, electrically evoked compound action potentials (ECAP) of electrically stimulated auditory nerve fibers are determined, and a dispersion of the ECAP magnitudes is used to estimate the noise signal. For example, the noise signal can be estimated based on a dispersion of residuals of a regression fit of the ECAP magnitudes, a dispersion of a difference between estimated ECAP magnitudes and short kernel median filtered ECAP magnitudes, and/or Equation 1, which provides a variance estimation of ECAP magnitudes using exponentially weighted means.

$$S \hat{} 2 = ( 1 - a ) * S \hat{} 2 * ( X - y ) \hat{} 2 \qquad Equation 1$$

In Equation 1, “S\hat{ }2” is the variance estimation of ECAP magnitudes, “a” is the smoothing factor with a period of about 2/(number of samples+1), “y” is a measured ECochG magnitude, and “X” is the approximate mean magnitude of ECochG signals and is equal to (1−a)*X+a*y. In further embodiments, frequency domain measurement signal-to-noise ratio (SNR) estimation, shown in Equation 2, can be performed.

$$SNR = 10 * log ( ( sum \vert fft ( k ) \vert \hat{} 2 ) / ( sum \vert fft ( j ) \vert \hat{} 2 ) ) \qquad Equation 2$$

In Equation 2, “k” contains expected frequency components of the ECochG signal multiplied by the period of the ECochG signal, “j” does not contain “0” or “k,” and “fft(x)” is the complex coefficient in “bin x” of the fast Fourier transformation (FFT) signal. In further embodiments, noise can be determined via assessment by FFT to define energy in frequency bins adjacent to a signal of interest. In any of these embodiments, the estimated noise signal is compared with the ECochG signals.

At 306, a determination is made regarding whether the ECochG signals are above the estimated noise signal, such as whether the ECochG signals exceed the estimated noise signal by a threshold amount. At 308, in response to a determination that the ECochG signals are above the estimated noise signal, a state of an inner ear is classified into an appropriate category based on the ECochG signals. For example, the ECochG signals being above the estimated noise signal indicates that the ECochG signals include detectable cochlea signals provided in response to the delivered acoustic stimuli. Thus, the ECochG signals can be further processed to classify the state of the inner ear. In some embodiments, classifying the state of the inner ear includes extracting CM magnitudes from the ECochG signals (e.g., via frequency analysis, such as bandpass filtering or fast Fourier transformation at the frequency of provided acoustic stimulus) and processing the CM magnitudes. In additional or alternative embodiments, other characteristics, such as a CM latency and/or an ANN, of the ECochG signals can be used to classify the state of the inner ear. At 312, feedback is output based on the classification of the state of the inner ear. The feedback that is output may notify a user, such as a surgeon, of the state of the inner ear to prompt the user to perform a corresponding action. Indeed, there are multiple categories in which the state of the inner ear can be classified, and different feedback corresponds to each category. As such, the specific feedback corresponding to the classified state of the inner ear is selected and provided to inform the user of the state of the inner ear.

However, at 310, in response to a determination that the ECochG signals are not above the estimated noise signal, a determination is made that no valid ECochG signals are received. That is, the ECochG signals being at or below the noise signal may indicate that the ECochG signals do not include a detectable cochlea signal provided in response to the delivered acoustic stimuli. Therefore, the received ECochG signals may not accurately represent the state of the inner ear (e.g., the ECochG signals may, instead, indicate received noise). In some embodiments, feedback can also be provided in response to the determination that no valid ECochG signals are received, as shown at 312. Thus, the feedback informs the user that no valid ECochG signals are received. Indeed, the feedback provided in response to a determination that no valid ECochG signals are received is different from any of the feedback provided based on a classification of the state of the inner ear in an appropriate category to distinguish that the state of the inner ear is not classifiable based on the ECochG signals. In additional or alternative embodiments, no feedback is provided in response to the determination that no valid ECochG signals are received. In this way, the lack of any feedback being provided indicates that the state of the inner ear is not classifiable based on the ECochG signals.

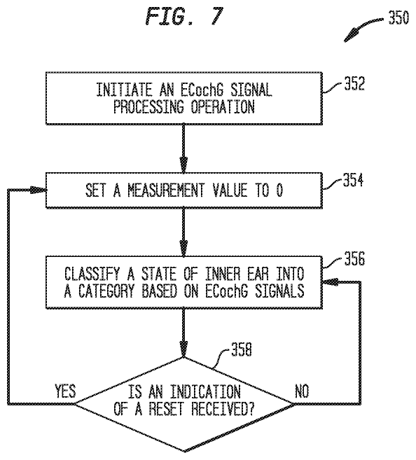

FIG. 7 is a flowchart of a method 350 of resetting a peak CM magnitude, in accordance with certain embodiments presented herein. At 352, an ECochG signal processing operation is initiated. For example, certain operations of the method 300 are performed in which ECochG signals are received and processed. At 354, measurement values of the ECochG signals are set to 0. As discussed herein, different measurements, such as CM magnitude, CM latency, and/or ANN, can be extracted from ECochG signals during processing of the ECochG signals. In some embodiments, during processing of the ECochG signals, certain values of the measurements (e.g., peak CM magnitude, minimum CM magnitude) are determined and used as reference for comparison with measurements of subsequently or other ECochG signals to classify a state of the inner ear. Upon initiation of the ECochG signal processing operation (e.g., prior to receipt of ECochG signals), the measurement values are set to 0 to prepare for receipt of the ECochG signals, adjustment of the measurement values from 0 based on the received ECochG signals, and processing based on the determined measurement values.

At 356, a state of the inner ear is classified into an appropriate category based on the received ECochG signals. For example, the aforementioned measurement values are updated (e.g., adjusted from 0) based on the received ECochG signals for comparison with additional ECochG signals. The state of the inner ear is then classified based on comparison of additional ECochG signals with the measurement values.

At 358, a determination is made regarding whether an indication of a reset of the ECochG signal processing operation is received. In some embodiments, the indication can be received via a user input, such as actuation of a feature (e.g., a user interface) of the ECochG monitoring system. In additional or alternative embodiments, the indication can be automatically received. For example, a reset can be automatically scheduled to occur at a particular time and/or at a specific frequency regardless of whether a user input is received. As another example, a reset can be identified based on receipt of other signals (e.g., sensed data). For instance, a reset is identified based on receipt of electrode impedances that indicate an open circuit corresponding to withdrawal of electrodes. In response to a determination that a reset is received, the measurement values are set to 0 again, as shown at 354. As such, upon receiving subsequent ECochG signals, the measurement values can be readily adjusted from 0. However, in response to a determination that an indication of a reset is not received, the state of the inner ear is classified without setting the measurement values to 0. Therefore, the measurement values that were previously set (e.g., based on previously received ECochG signals) are maintained, and subsequent ECochG signals can be compared to the previously set measurement values to classify the state of the inner ear. Indeed, because the measurement values are not reset to 0, the measurement values may not be changed based on the subsequent ECochG signals.

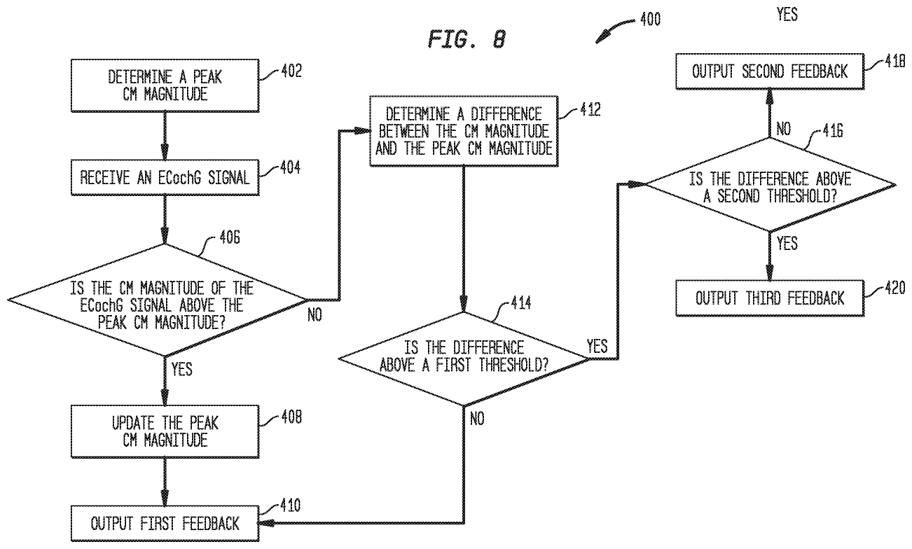

FIG. 8 is a flowchart of a method 400 of providing feedback based on CM magnitudes, in accordance with certain embodiments presented herein. At 402, a peak CM magnitude is determined. For example, the peak CM magnitude is determined by extracting CM magnitudes from initially received ECochG signals and selecting the highest CM magnitude that was extracted. At 404, an ECochG signal (e.g., a subsequently received ECochG signal) is received as a result of delivering an acoustic stimulus to a region of the inner ear, and the CM magnitude of the received ECochG signal is extracted. At 406, a determination is made regarding whether the CM magnitude of the received ECochG signal is above the peak CM magnitude.

At 408, in response to a determination that the CM magnitude of the received ECochG signal is above the peak CM magnitude, the peak CM magnitude is updated. In particular, the value of the peak CM magnitude is updated to be the CM magnitude of the received ECochG signal. At 410, first feedback is output to indicate classification of the state of the inner ear into a first category in which the CM magnitude is high (e.g., greater than the previously received ECochG signals used to determine the initial peak CM magnitude). For instance, the first category indicates the region of the inner ear includes an expected or desirable amount of functioning hair cells (e.g., outer hair cells).

However, at 412, in response to a determination that the CM magnitude of the received ECochG signal is not above the peak CM magnitude (e.g., CM magnitudes are decreasing with respect to the previously received ECochG signals used to determine the initial peak CM magnitude), a difference between the CM magnitude and the peak CM magnitude is determined. At 414, a determination is made regarding whether the difference is above a first threshold, which may be a relatively smaller value (e.g., 10% of the peak CM magnitude). In response to a determination that the difference is above the first threshold, the first feedback is output, as shown at 410. In this way, the first category indicated by the first feedback can also include a state of the inner ear in which the CM magnitudes are stable and about the same as the peak CM magnitude (e.g., CM magnitudes are not decreasing at a noticeable rate).

At 416, in response to a determination that the difference is above the first threshold, another determination is made regarding whether the difference is above a second threshold, which is a relatively larger value (e.g., 30% of the peak CM magnitude). At 418, in response to a determination that the difference is not above the second threshold, second feedback is provided to indicate classification of the state of the inner ear into a second category in which the CM magnitude is somewhat low. For example, the region of the inner ear may not have an expected or desirable amount of functioning hair cells and/or the amount of functioning hair cells may be decreasing, but the amount of functioning hair cells may still be sufficient. Thus, the second feedback can notify a user to monitor upcoming ECochG signals to be received or to perform any other suitable action to prepare to potentially address the low CM magnitude (e.g., somewhat decreasing CM magnitudes) based on the upcoming ECochG signals to be received.

At 420, in response to a determination that the CM magnitude is above the second threshold, third feedback is provided. The third feedback indicates classification of the state of the inner ear into a third category in which CM magnitude is significantly low and that action is to be performed (e.g., performed immediately). For example, the region of the inner ear does not include a sufficient amount of functioning hair cells, and the third feedback can prompt a user to perform an action to address the low CM magnitudes (e.g., significantly decreasing CM magnitudes).

The first feedback, the second feedback, and the third feedback can be any combination of audio feedback, visual feedback, and/or tactile feedback. Additionally, the first feedback, the second feedback, and the third feedback are different from one another to indicate different categories, such as different urgencies. For instance, the first feedback can indicate the least amount of urgency, and the third feedback can indicate the most amount of urgency. In an example, the first feedback can include audio tones output at a relatively slow rate, at a relatively low intensity, and/or at a relatively low frequency (e.g., 400 Hz), the second feedback can include audio tones output at a relatively medium rate, at a relatively medium intensity, and/or at a relatively medium frequency (e.g., 600 Hz), and the third feedback can include audio tones output at a relatively fast rate, at a relatively high intensity, and/or at a relatively high frequency (e.g., 800 Hz). In another example, the first feedback can include a light output at a relatively low intensity and/or at a relatively low flashing frequency, the second feedback can include light output at a relatively medium intensity and/or at a relatively medium frequency, and the third feedback can include light output at a relatively high intensity and/or at a relatively high frequency. In a further example, the first feedback can include a vibration output at a relatively slow rate and/or at a relatively low intensity, the second feedback can include a vibration output at a relatively medium rate and/or at a relatively medium intensity, and the third feedback can include a vibration output at a relatively high rate and/or at a relatively high intensity. Indeed, the first feedback, the second feedback, and the third feedback can have any distinguishable differences in characteristics, such as different colors of light, different displayed symbols, different sounds, different haptic outputs (e.g., vibration versus temperature change), different types of feedback, and so forth, to indicate classification of the state of the inner ear into different categories. In any of these examples, the third feedback can potentially capture the attention of a user more quickly to prompt the user to take more immediate action to mitigate the decreasing CM magnitudes.

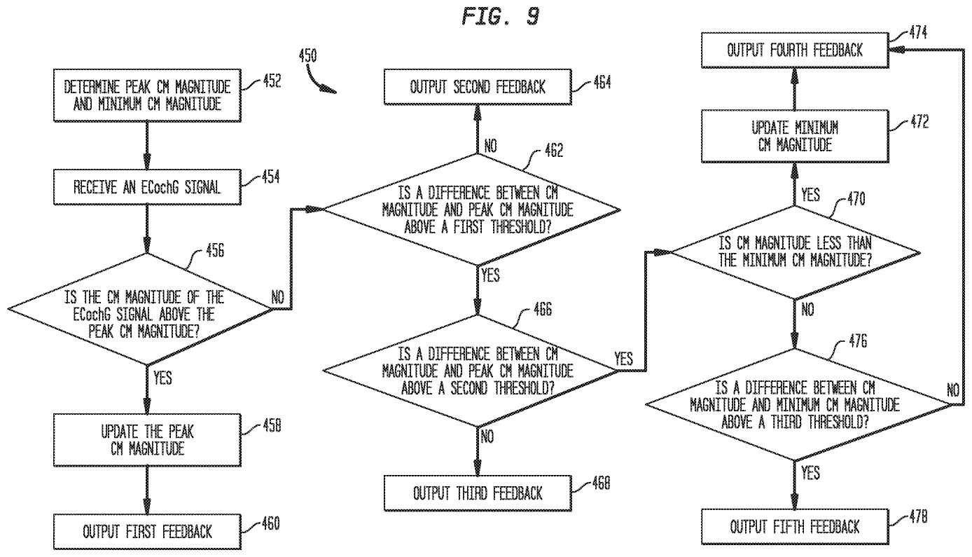

FIG. 9 is a flowchart of a method 450 of providing feedback based on CM magnitudes, in accordance with certain embodiments presented herein. At 452, a peak CM magnitude and a minimum CM magnitude are determined. Each of the peak CM magnitude and the minimum CM magnitude can be determined from initially received ECochG signals by selecting the highest CM magnitude extracted from the initially received ECochG signals as the peak CM magnitude and the lowest CM magnitude extracted from the initially received ECochG signals as the minimum CM magnitude. At 454, an ECochG signal is received as a result of delivering an acoustic stimulus to a region of the inner ear, and the CM magnitude of the received ECochG signal is extracted. At 456, a determination is made regarding whether the CM magnitude of the ECochG signal is above the peak CM magnitude.

At 458, in response to a determination that the CM magnitude of the received ECochG signal is above the peak CM magnitude, the peak CM magnitude is updated to be that of the CM magnitude of the received ECochG signal. At 460, first feedback is output to indicate classification of the state of the inner ear into a first category in which the CM magnitudes are high, such as the amount of functioning hair cells (e.g., outer hair cells) across the region of the inner ear being expected or desirable and increasing.

At 462, in response to a determination that the CM magnitude of the received ECochG signal is not above the peak CM magnitude, a further determination is made regarding whether a difference between the CM magnitude and the peak CM magnitude is above a first threshold, which is a relatively smaller value. At 464, in response to a determination that the difference is not above the first threshold, second feedback is output to indicate classification of the inner ear into a second category in which the CM magnitudes are about the same as the peak CM magnitude, such as the region of the inner ear including an expected or desirable, but not noticeably increasing, amount of functioning hair cells.

At 466, in response to a determination that the difference between the CM magnitude and the peak CM magnitude is above the first threshold, another determination is made regarding whether the difference is above a second threshold, which is a relatively larger value. At 468, in response to a determination that the difference is not above the second threshold value, third feedback is output to indicate classification of the state of the inner ear into a third category in which the CM magnitude is somewhat low, such as the region of the inner ear including an unexpected or undesirable, but still sufficient, amount of functioning hair cells.

At 470, in response to a determination that the difference between the CM magnitude and the peak CM magnitude is above the second threshold (e.g., the CM magnitude is substantially lower than the peak CM magnitude), an additional determination is made regarding whether the CM magnitude is less than the minimum CM magnitude. At 472, in response to a determination that the CM magnitude is less than the minimum CM magnitude, the minimum CM magnitude is updated to be the CM magnitude of the ECochG signal. At 474, fourth feedback is then output to indicate classification of the state of the inner ear into a fourth category in which the CM magnitude is significantly low, such as the region of the inner ear including an insufficient amount of functioning hair cells.

At 476, in response to a determination that the CM magnitude is less than the minimum CM magnitude, a determination is made regarding whether the difference between the CM magnitude and the minimum CM magnitude is above a third threshold. In response to a determination that the difference between the CM magnitude and the minimum CM magnitude is not above the third threshold (e.g., the CM magnitude is about as low as the minimum CM magnitude), the fourth feedback is output to indicate classification of the state of the inner ear into the fourth category in which the CM magnitude is significantly low.

However, at 478, in response to a determination that the difference between the CM magnitude and the minimum CM magnitude is above the third threshold, fifth feedback is output. The fifth feedback indicates classification of the state of the inner ear into a fifth category in which the CM magnitude is substantially lower than the peak CM magnitude, but also substantially higher than the minimum CM magnitude. For example, the fifth category indicates improving responses to delivered acoustic stimuli at the region of the inner ear, such as the region of the inner ear including a recovering amount of functioning hair cells (e.g., the amount of functioning hair cells is low, but recovering).

In some embodiments, a new peak CM magnitude can be set based on ECochG signals newly received after classification of the state of the inner ear into the fifth category. By way of example, a determination is made that a normal or stable response state of the inner ear is reached based on the slope of the CM magnitudes of the newly received ECochG signals being stable and/or based on a difference between the CM magnitudes of the newly received ECochG signals and the previously determined peak CM magnitude being less than the first threshold, either of which can indicate the ECochG responses have recovered. A new peak CM magnitude can then be determined based on a maximum of the CM magnitudes of the newly received ECochG signals, even if the new peak CM magnitude is less than the previously determined peak CM magnitude.

The feedback provided via the method 450 can indicate more specific states of the inner ear as compared to the feedback provided via the method 400. As such, a user can potentially determine a more suitable action to be performed based on the specific state of the inner ear indicated by provided feedback. Indeed, the first feedback, the second feedback, the third feedback, the fourth feedback, and the fifth feedback, which can include any combination of audio feedback, visual feedback, and/or tactile feedback, are different from one another to enable the feedbacks to be easily distinguished from one another (e.g., to indicate different urgencies). By way of example, the first feedback can include audio tones output at a relatively slow rate and at a relatively low frequency (e.g., 400 Hz), the second feedback can include audio tones output at a relatively slow rate and at a relatively medium frequency (e.g., 600 Hz), or at a relatively medium rate and at a relatively low frequency, the third feedback can include audio tones output at a relatively medium rate and at a relatively medium frequency, the fourth feedback can include audio tones output at a relatively high rate and a relatively high frequency (e.g., 800 Hz), and the fifth feedback can include audio tones output at a relatively medium rate and at a relatively high frequency, or at a relatively high rate and at a relatively medium frequency. The different permutations of the characteristics of feedback (e.g., rate, frequency, intensity, duration, type of feedback) can indicate the different classified states of the inner ear.

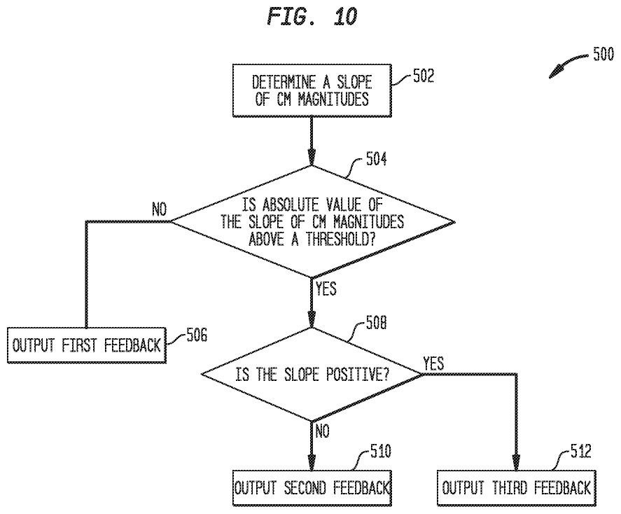

FIG. 10 is a flowchart of a method 500 of providing feedback based on CM magnitudes, in accordance with certain embodiments presented herein. At 502, a slope of CM magnitudes of received ECochG signals is determined. The slope can be determined via a linear regression technique (e.g., for three or more CM magnitudes). At 504, a determination is made regarding whether an absolute value of the slope is above a threshold. At 506, in response to a determination that the absolute value of the slope is above the threshold, first feedback is output to indicate classification of the state of the inner ear into a first category in which the CM magnitudes are relatively stable (e.g., CM magnitudes are not noticeably increasing or decreasing).

However, at 508, in response to a determination that the absolute value of the slope is not above the threshold, a further determination is made regarding whether the slope is positive. At 510, second feedback is output to indicate classification of the state of the inner ear into a second category in which the CM magnitudes are increasing in response to a determination that the slope is positive. At 512, third feedback is output to indicate classification of the state of the inner ear into a third category in which the CM magnitude is decreasing.

In this manner, based on the feedback being output, a user can determine the trend of the CM magnitudes. The feedback can include a change in characteristic that corresponds to the trend of the CM magnitudes. As an example, the first feedback indicative of relatively stable CM magnitudes can include an audio tone, a light, and/or a vibration that is output at a constant rate, a constant frequency, at a constant intensity, the second feedback indicative of decreasing CM magnitudes can include an audio tone, a light, and/or a vibration that is output at a decreasing rate, a decreasing frequency, and/or at a decreasing intensity, and the third feedback indicative of increasing CM magnitudes can include an audio tone, a light, and/or a vibration that is output at an increasing rate, an increasing frequency, and/or at an increasing intensity. In this way, the user can more readily predict or determine potential subsequent CM magnitudes and prepare to perform a suitable action based on provided feedback.

It should be noted that, in accordance with any of the methods 350, 400, 450 described herein, the feedback being output can have multiple different characteristics to indicate granular states of the inner ear, such as to indicate both a single measurement and a trend of the ECochG signals. For example, audio tones being output can have both a high frequency (e.g., based on the CM magnitude being significantly low) and also a decreasing frequency (e.g., based on multiple CM magnitudes decreasing). As such, a user can perform an action more suitably as compared to feedback that indicates a single measurement or a trend, but not both.

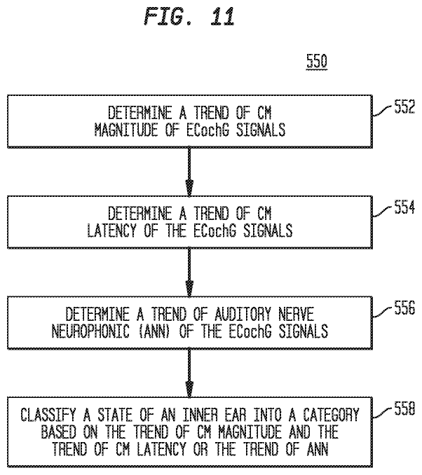

FIG. 11 is a flowchart of a method 550 for classifying a state of an inner ear using different measurements of ECochG signals, in accordance with embodiments presented herein. At 552, a trend (e.g., a slope) of CM magnitude of ECochG signals is determined. At 554, a trend of CM latency of the ECochG signals is determined. At 556, a trend of ANN of the ECochG signals is determined. The trends of the CM magnitudes, of the ECochG signals, and/or of the ANN are the compared with one another. At 558, the state of the inner ear is classified into a category based on the trend of the CM magnitude and at least one of the trend of the CM latency or the trend of the ANN. In other words, the state of the inner ear is classified based on a combination of the CM magnitude trend, the CM latency trend, and the ANN trend, which can provide a more accurate and/or specific classification of the state of the inner ear. In some embodiments, determination of the trend of ANN and/or of CM latency is selectively invoked based on the trend of CM magnitude. For example, the trend of ANN determined in response to a determination that the CM magnitude is decreasing and/or the trend of CM latency is determined in response to a determination that the CM magnitude is changing. Alternatively, the trend of ANN and/or of CM latency is continually determined regardless of the determined trend of CM magnitude.

As an example, the trend of the ANN can be used to verify loss or absence of functioning hair cells. For instance, insufficient hair cells (e.g., insufficient cochlea response to delivered acoustic stimuli) should cause both the CM magnitude and the ANN to decrease. Thus, in response to a determination that both the CM magnitude and the ANN are decreasing (e.g., both CM magnitude and ANN are below 30% of their respective peak values), the state of the inner ear is classified in a first category in which the amount of hair cells is decreasing or low, and corresponding feedback is provided to indicate classification of the state of the inner ear into the first category. However, in response to a determination that the CM magnitude is decreasing, and the ANN is not decreasing (e.g., the ANN is steady or increasing), such as that the ratio between ANN values and CM magnitude values is increasing, the state of the inner ear is not classified into the first category. Instead, for example, the state of the inner ear can be classified into a second category in which the amount of functioning hair cells is sufficient or in which the state of the inner ear is indeterminable, and corresponding feedback is provided. In some embodiments, the ANN values or the changes in ANN values are relatively small. Therefore, the ANN is compared to an estimated noise signal to determine whether the trend of ANN can be reliably used and determined (e.g., to indicate the state of the inner ear, rather than to indicate a change in noise interference).

As another example, CM latency of ECochG signals (e.g., during insertion or between electrodes) in combination with CM amplitude can be used to determine even more specific states of the inner ear. For instance, in response to a determination that the CM magnitude is increasing, and the CM latency is stable or constant, the state of the inner ear is classified into a first category in which there is an absence of functioning hair cells across the corresponding region of the inner ear. In response to a determination that the CM magnitude is increasing, and the CM latency is increasing, the state of the inner ear is classified into a second category in which the amount of functioning hair cells across the corresponding region of the inner ear is sufficient. In response to a determination that the CM magnitude is decreasing, and the CM latency is stable (e.g., after initially increasing), the state of the inner ear is classified into a third category in which the amount of functioning hair cells is decreasing. In response to a determination that the CM magnitude is decreasing, and the CM latency is increasing, the state of the inner ear is classified into a fourth category in which functioning hair cells are present, but have reduced ability to perceive the provided acoustic stimulus (e.g., the characteristic frequency of the acoustic stimulus is too high).

Such specific states can further prompt the user to perform a suitable action. For example, based on classification of the state of a particular region of the inner ear in the first category in which there is an absence of functioning hair cells, the user determines that the inner ear is inherently non-functioning beyond the particular region and that the insertion depth is to be established at the particular region to provide sufficient stimulation and enable perception of sound. That is, by combining the information about the insertion depth and the ECochG signals, a user can better interpret changes to the ECochG signals, such as changes to the CM magnitude.

By way of example, a target insertion depth (e.g., a minimum target insertion depth) can be determined, such as based on a relative amount of functioning hair cells, as indicated by the ECochG signals, at different regions of the inner ear, as indicated by the corresponding insertion depth. Indeed, a user can more suitably determine the target insertion depth based on ECochG responsiveness at the target insertion depth and the coverage provided at the target insertion depth to provide electrical stimulation for hearing. Additionally, based on classification of the state of the inner ear into the fourth category in which functioning hair cells are present and have reduced ability to perceive the provided acoustic stimulus, the user can determine the operation of the stimulating assembly is to be adjusted to change (e.g., lower) the frequency of the provided acoustic stimulus and enable the functioning hair cells to respond. However, based on classification of the state of the inner ear into the third category in which the amount of functioning hair cells is decreasing, the user determines that reduced function of the inner ear is caused by undesirable positioning of the stimulating assembly (e.g., as a result of electrode contact with the basilar membrane), and the user can determine to adjust the positioning of the stimulating assembly. In this manner, based on the category in which the state of the inner ear is classified, the user is able to determine a cause or reason for the state of the inner ear (e.g., a region that includes reduced or absence of functioning hair cells) to perform a more suitable action.

Moreover, information provided by ECochG signals can be useful after implantation is performed. As an example, CM magnitude and CM latency recorded for different regions of the inner ear during implantation can be stored and referenced to determine the specific regions of the inner ear having functioning hair cells. Such regions can then be used to determine a potential cross-over point of the inner ear at which acoustic stimulation is used before the cross-over point and electrical stimulation is provided beyond the cross-over point for implementing an electrical acoustic stimulation system in which both acoustic stimulation (e.g., via a hearing aid) and electrical stimulation (e.g., via a cochlear implant) are to be provided. As another example, particular acoustic stimuli can be delivered to invoke ECochG signals during or after implantation to detect peripheral nerve survival at specific regions in the inner ear. The characteristic of electrical stimulation at different, specific regions can then be established accordingly, thereby providing a more personalized and suitable stimulation operation for the recipient based on the inner ear functions specific to the recipient.

Certain other parameters can also be used to classify the state of the inner ear. For example, in response to detected SP (e.g., during implantation, after implantation), the state of the inner ear is classified to indicate functioning hair cells (e.g., inner hair cells) that provide support for auditory nerves as targets for electrical stimulation. Thus, other biomarkers can be used in conjunction with the parameters discussed herein to classify the state of the inner ear into different categories, such as specific categories that are unable to be detected based on CM magnitude alone.

Moreover, it should be noted that in addition to or as an alternative to providing different feedback to indicate the state of the inner ear, different measurement values of the ECochG signals (e.g., the CM magnitude, the CM latency, the ANN) can be displayed. For example, a digital microscope can superimpose plots of the measurement values on an edge of a surgical field to be more readily observable. As such, the user can utilize the measurement values to determine the state of the inner ear (e.g., to confirm the feedback provided corresponds to the measurement values).

It should also be noted that for any of the described methods 300, 350, 400, 450, 500, 550, machine learning can be utilized to adjust classification of the state of the inner ear into an appropriate category. By way of example, the state of the inner ear is classified into an initial category based on received ECochG signals (e.g., the CM magnitude of the ECochG signals) by using a data model (e.g., an algorithm), and an input (e.g., a user input) is provided to indicate whether the initial category in which the state of the inner ear is classified is accurate for the ECochG signals. In response to receiving an input confirming that the initial category is accurate, the data model used to classify the state of the inner ear can be reinforced to enable classification of the state of the inner ear into the initial category based on receipt of the same or similar ECochG signals (e.g., ECochG signals having similar CM magnitude values or trends). However, in response to receiving an input indicating that the initial category is inaccurate, the data model used to classify the state of the inner ear can be adjusted to cause classification of the state of the inner ear into an adjusted category (e.g., an accurate category) based on receipt of the same or similar ECochG signals. As such, the manner in which ECochG signals are analyzed can be adjusted to classify the state of the inner ear more accurately into an appropriate category.

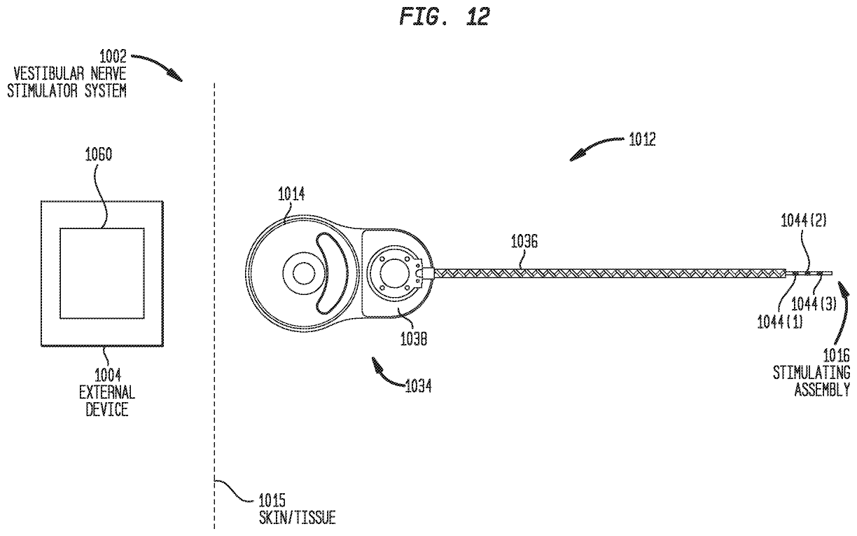

As previously described, the technology disclosed herein can be applied in any of a variety of circumstances and with a variety of different devices. Example devices that can benefit from technology disclosed herein are described in more detail in FIG. 12. The techniques of the present disclosure can be applied to other devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 12 illustrates an example vestibular stimulator system 1002, with which embodiments presented herein can be implemented. As shown, the vestibular stimulator system 1002 comprises an implantable component (vestibular stimulator) 1012 and an external device/component 1004 (e.g., external processing device, battery charger, remote control, etc.). The external device 1004 comprises a transceiver unit 1060. As such, the external device 1004 is configured to transfer data (and potentially power) to the vestibular stimulator 1012,

The vestibular stimulator 1012 comprises an implant body (main module) 1034, a lead region 1036, and a stimulating assembly 1016, all configured to be implanted under the skin/tissue (tissue) 1015 of the recipient. The implant body 1034 generally comprises a hermetically-sealed housing 1038 in which RF interface circuitry, one or more rechargeable batteries, one or more processors, and a stimulator unit are disposed. The implant body 134 also includes an internal/implantable coil 1014 that is generally external to the housing 1038, but which is connected to the transceiver via a hermetic feedthrough (not shown).

The stimulating assembly 1016 comprises a plurality of electrodes 1044(1)-(3) disposed in a carrier member (e.g., a flexible silicone body). In this specific example, the stimulating assembly 1016 comprises three (3) stimulation electrodes, referred to as stimulation electrodes 1044(1), 1044(2), and 1044(3). The stimulation electrodes 1044(1), 1044(2), and 1044(3) function as an electrical interface for delivery of electrical stimulation signals to the recipient's vestibular system.

The stimulating assembly 1016 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the vestibular stimulator 1012, the external device 1004, and/or another external device, can be configured to implement the techniques presented herein. That is, the vestibular stimulator 1012, possibly in combination with the external device 1004 and/or another external device, can include an evoked biological response analysis system, as described elsewhere herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. One or more non-transitory computer readable media, comprising instructions that, when executed by processing circuitry, cause the processing circuitry to:
receive an electrocochleography (ECochG) signal associated with a recipient;
classify a state of an inner ear of the recipient into an appropriate category of a plurality of categories based on at least one characteristic of the ECochG signal; and
output feedback in response to classifying the state of the inner ear into the appropriate category.

2. The one or more non-transitory computer readable media of claim 1, wherein each category of the plurality of categories corresponds to a respective feedback of a plurality of feedback, and wherein the instructions, when executed by the processing circuitry, cause the processing circuitry to:
select the feedback from the plurality of feedback based on the feedback corresponding to the appropriate category of the plurality of categories; and
output the feedback selected from the plurality of feedback.

3. The one or more non-transitory computer readable media of claim 1, wherein the at least one characteristic of the ECochG signal comprises a cochlear microphonic (CM) magnitude, and wherein the instructions, when executed by the processing circuitry, cause the processing circuitry to:
compare the CM magnitude to a peak CM magnitude associated with at least one previously received ECochG signal; and
classify the state of the inner ear of the recipient into a first category of the plurality of categories in response to determining the CM magnitude is above the peak CM magnitude.

4. The one or more non-transitory computer readable media of claim 3, wherein the feedback is first feedback, and wherein the instructions, when executed by the processing circuitry, cause the processing circuitry to:
determine a difference between the CM magnitude and the peak CM magnitude in response to determining the CM magnitude is not above the peak CM magnitude;
classify the state of the inner ear of the recipient into a second category of the plurality of categories in response to determining the difference between the CM magnitude and the peak CM magnitude is above a threshold; and
output second feedback in response to classifying the state of the inner ear into the second category.

5. The one or more non-transitory computer readable media of claim 4, wherein the threshold is a first threshold, and wherein the instructions, when executed by the processing circuitry, cause the processing circuitry to:
compare the difference between the CM magnitude and the peak CM magnitude to a second threshold, greater than the first threshold;
classify the state of the inner ear of the recipient into the second category of the plurality of categories in response to determining the difference between the CM magnitude and the peak CM magnitude is above the first threshold and the second threshold;
classify the state of the inner ear of the recipient into a third category of the plurality of categories in response to determining the difference between the CM magnitude and the peak CM magnitude is between the first threshold and the second threshold; and
output third feedback in response to classifying the state of the inner ear into the third category.

6. The one or more non-transitory computer readable media of claim 4, wherein the instructions, when executed by the processing circuitry, cause the processing circuitry to:
classify the state of the inner ear of the recipient into a third category of the plurality of categories in response to determining the difference between the CM magnitude and the peak CM magnitude is not above the threshold; and
output third feedback in response to classifying the state of the inner ear into the third category.

7. The one or more non-transitory computer readable media of claim 4, wherein the instructions, when executed by the processing circuitry, cause the processing circuitry to:
compare the CM magnitude with a minimum CM magnitude associated with the at least one previously received ECochG signal in response to determining the difference between the CM magnitude and the peak CM magnitude is above the threshold; and
classify the state of the inner ear of the recipient into the second category of the plurality of categories in response to determining the CM magnitude is less than the minimum CM magnitude.

8. The one or more non-transitory computer readable media of claim 7, wherein the instructions, when executed by the processing circuitry, cause the processing circuitry to:
determine a difference between the CM magnitude and the minimum CM magnitude in response to determining the CM magnitude is not less than the minimum CM magnitude;
classify the state of the inner ear of the recipient into a third category of the plurality of categories in response to determining the difference between the CM magnitude and the minimum CM magnitude is above an additional threshold; and
output third feedback in response to classifying the state of the inner ear into the third category.

9. The one or more non-transitory computer readable media of claim 8, wherein the instructions, when executed by the processing circuitry, cause the processing circuitry to:
classify the state of the inner ear of the recipient into the second category of the plurality of categories in response to determining the difference between the CM magnitude and the minimum CM magnitude is not above the additional threshold.

10. The one or more non-transitory computer readable media of claim 1, wherein the feedback comprises at least one of audio feedback, visual feedback, or tactile feedback.

11. A method, comprising:
delivering acoustic stimuli to an ear of a recipient;
recording a plurality of electrocochleography (ECochG) signals evoked at the ear of the recipient in response to delivery of the acoustic stimuli; and
classifying a state of an inner ear of the recipient into an appropriate category of a plurality of categories based on a characteristic of the plurality of ECochG signals.

12. The method of claim 11, wherein the characteristic of the plurality of ECochG signals comprises a plurality of cochlear microphonic (CM) magnitudes, and the method comprises:
determining a slope of the CM magnitudes; and
classifying the state of the inner ear of the recipient into the appropriate category of the plurality of categories based on slope of the CM magnitudes.

13. The method of claim 12, comprising:
comparing an absolute value of the slope of the CM magnitudes to a threshold; and
classifying the state of the inner ear into the appropriate category based on comparison of the absolute value of the slope to the threshold.

14. The method of claim 13, comprising:
classifying the state of the inner ear into a first category of the plurality of categories in response to determining the absolute value of the slope of the CM magnitudes is not above the threshold.

15. The method of claim 13, comprising:
determining whether the slope is positive in response to determining the absolute value of the slope of the CM magnitudes is above the threshold; and
classifying the state of the inner ear into the appropriate category in response to determining whether the slope is positive.

16. (canceled)

17. (canceled)

18. A system, comprising:
an output device;
a network interface for communication with a medical device comprising a plurality of electrode contacts;
a memory storing instructions thereon; and
processing circuitry configured to execute the instructions stored on the memory to:
receive a plurality of electrocochleography (ECochG) signals during implantation of the plurality of electrode contacts into a recipient;
classify a state of an inner ear of the recipient into an appropriate category of a plurality of categories based on a characteristic of the plurality of ECochG signals, wherein each category of the plurality of categories is associated with respective feedback of a plurality of feedback; and
instruct the output device to output appropriate feedback of the plurality of feedback based on the appropriate feedback corresponding to the appropriate category.

19. The system of claim 18, wherein the characteristic of the ECochG signals comprises a plurality of cochlear microphonic (CM) magnitudes, and wherein the processing circuitry is configured to execute the instructions stored on the memory to:
determine a trend of the plurality of CM magnitudes; and
classify the state of the inner ear of the recipient into the appropriate category based on the trend of the plurality of CM magnitudes.

20. The system of claim 19, wherein the processing circuitry is configured to execute the instructions stored on the memory to:
determine a plurality of auditory nerve neurophonics (ANNs) from the plurality of ECochG signals; and
classify the state of the inner ear of the recipient into the appropriate category based on the trend of the plurality of CM magnitudes and a trend of the plurality of ANNs.

21. The system of claim 20, wherein the processing circuitry is configured to execute the instructions stored on the memory to:
classify the state of the inner ear of the recipient into a selected category of the plurality of categories in response to determining the trend of the plurality of CM magnitudes is decreasing and the trend of the plurality of ANNs is decreasing.

22. (canceled)

23. The system of claim 19, wherein the processing circuitry is configured to execute the instructions stored on the memory to:
determine a trend of a plurality of CM latencies from the plurality of ECochG signals; and
classify the state of the inner ear of the recipient into the appropriate category based on the trend of the plurality of CM magnitudes and a trend of the plurality of CM latencies.

24-27. (canceled)
